(19) United States
(12) Patent Application Publication
(10) Pub. No.: US 2006/0083705 A1
(43) Pub. Date: Apr. 20, 2006

(54) Vinyl lactam/vinyl carbazole copolymers

(75) Inventors: Janusz Jachowicz, Bethel, CT (US); Jui-Chang Chuang, Wayne, NJ (US); Roger L. Mc Mullen JR., West New York, NJ (US); Thomas Winkler, Maywood, NJ (US)

Correspondence Address: Attn: William J. Davis, Esq., Legal Department;INTERNATIONAL SPECIALTY PRODUCTS, Building No. 8, 1361 Alps Road, Wayne, NJ, 07470, US

(73) Assignee: ISP INVESTMENTS INC.

(21) Appl. No.: 11/252,354

(22) Filed: Oct. 17, 2005

Related U.S. Application Data
(60) Provisional application No. 60/620,196, filed on Oct. 19, 2004.

(51) Int. Cl. A61K 8/81

(52) U.S. Cl. 424/70.15

(57) ABSTRACT

A copolymer of a vinyl lactam and vinyl carbazole useful in personal care compositions.

### CROSS REFERENCE TO RELATED PATENT APPLICATION

The present application claims benefit of U.S. Provisional Application No. 60/620,196 filed Oct. 19, 2004.

### BACKGROUND OF THE INVENTION

This invention relates to polymers useful for personal care products, and, more particularly, to vinyl lactam/vinyl carbazole copolymers having advantageous properties for skin and hair care applications.

### SUMMARY OF THE INVENTION

Copolymers of vinyllactam, e.g. vinyl pyrrolidone or vinyl caprolactam, and vinyl carbazole were prepared by solution polymerization in ethanol. The structure of the vinyl pyrrolidone/vinyl carbazole copolymer is shown below:

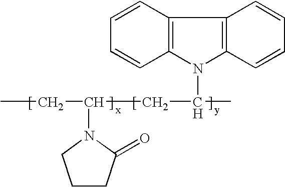

vinyl pyrrolidone/vinyl carbazole (VP/VCZ) copolymer

The experimental procedures are given in Examples 1 and 2 below. The copolymers of vinyl pyrrolidone with a vinyl carbazole content of 10 and 25% w/w are water soluble and alcohol soluble, respectively. The copolymers are characterized by UV absorption with the maximum at 360 nm (Example 3) and fluorescence emission with maximums at 390 nm and 420 nm.

A variety of copolymers, terpolymers and tetrapolymers based on vinyl pyrrolidone or vinyl caprolactam and carbazole with one or more other monomers, e.g. dimethylaminopropyl(meth)acrylamide (DMAPMA), C₁-C₈-quaternized DMAPMA, dimethylaminoethyl(meth)acrylate (DMAEMA), C₁-C₁₈-quaternized DMAEMA, and acrylamide, also are suitable in the invention. They are preferably characterized by visible light fluorescence which enhances luster (or add glitter, sparkle, glisten, or luminance) to hair treated with products containing them, which is a very attractive feature of hair conditioners or hair fixatives such as gels, mousses, or hairsprays.

The main applications for these copolymers are for personal care products, e.g. hair and skin treatments. Hair care applications include hair styling products, leave-in hair conditioners and hair shiners. Skin care applications include moisturizers, sunscreens, treatment lotions, etc.

The copolymers were tested as a treatment of hair for the improvement of luster (Example 4). It was shown that VP/VCZ copolymers are characterized by superior reflective properties as compared to a PVP homopolymer (Example 4).

The UV spectra of VP/VCZ copolymers indicates they act as UVB and UVA photo-protectors for hair and skin.

Based on UV and fluorescence spectra, the VP/VCZ copolymers are useful in skin care products for masking wrinkles.

### DESCRIPTION OF THE DRAWINGS

FIG. 1 shows the UV-VIS spectrum of vinyl pyrrolidone/vinyl carbazole copolymer in aqueous solution at a concentration of 1 g/l.

FIG. 2 shows the width of a specular reflection peak for hair treated with various polymers.

FIG. 3 shows the intensity of a specular reflection peak for hair treated with various polymers.

FIG. 4 shows the Reich-Robbins luster parameter for hair treated with various polymers.

FIG. 5 shows the Stamm luster parameter for hair treated with various polymers.

### DETAILED DESCRIPTION OF THE INVENTION

### EXAMPLE 1

### Preparation of Vinylpyrrolidone/9-Vinylcarbazole (90/10) Copolymer in Ethanol

Into a one-liter, four-necked, jacketed glass reactor, fitted with an anchor-type agitator, a nitrogen inlet tube, a thermometer and a reflux condenser, charge 144 g of vinylpyrrolidone, 16 g of 9-vinylcarbazole and 160 g of anhydrous ethanol. The reactants were bubbled with nitrogen for 15 minutes, followed by heating them to the reaction temperature of 78° C. The first initiator, 0.80 g of 20.0% 2,2′-azobis(methylbutyronitrile) (VAZO® 67 from Du Pont) in ethanol, was then added at time t=0. After reaching a mild exotherm peak of 87° C. in 15 minutes, the batch was controlled to 78° C. and maintained throughout the experiment. The second VAZO® 67 initiator of same amount was added at time t=1 hour from start and the batch was held for 2 hours at 78° C. At this point the polymer solution at 50% solids became very viscous and was diluted with 80.0 g of anhydrous ethanol to 40% solids. Three booster initiators (1.60 g of 20.0% VAZO® 67 in ethanol each) were added at time t=4, 8 and 12 hours, respectively. After holding the batch for 4 hours, it was cooled to ambient temperature and discharged into a glass bottle. The polymer solution is very slightly hazy and is soluble in water.

### EXAMPLE 2

### Preparation of Vinylpyrrolidone/9-Vinylcarbazole (75/25) Copolymer in Ethanol

Into a one-liter, four-necked, jacketed glass reactor, fitted with an anchor-type agitator, a nitrogen inlet tube, a thermometer and a reflux condenser, charge 120 g of vinylpyrrolidone, 40 g of 9-vinylcarbazole and 160 g of anhydrous ethanol. The reactants were bubbled with nitrogen for 15 minutes, followed by heating them to the reaction temperature of 78° C. The first initiator, 0.80 g of 20.0% 2,2′-azobis(methylbutyronitrile) (VAZO® 67 from Du Pont) in ethanol, was then added at time t=0. After reaching a mild exotherm peak of 88° C. in 15 minutes, the batch was controlled to 78° C. and maintained throughout the experiment. The second VAZO® 67 initiator of same amount was added at time t=1 hour from start and the batch was held for 2 hours at 78° C. At this point the polymer solution at 50% solids became very viscous and was diluted with 80.0 g of anhydrous ethanol to 40% solids. Three booster initiators (1.60 g of 20.0% VAZO® 67 in ethanol each) were added at time t=4, 8 and 12 hours, respectively. The batch was held at 78° C. for 4 hours after the addition of the third booster. The batch was then cooled to ambient temperature and discharged into a glass bottle. The polymer solution is hazy in ethanol and is insoluble in water.

### EXAMPLE 3

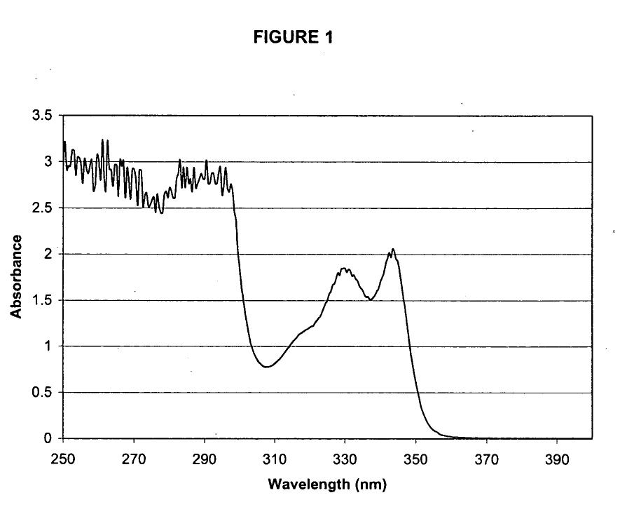

UV-VIS spectrum of vinyl pyrrolidone/vinyl carbazole copolymer in aqueous solution at a concentration of 1 g/l is shown in FIG. 1.

The copolymer is characterized by the maximum absorbance at 330 nm and 345 nm in the UVA range and 290 nm in the UVB range of solar emission.

### EXAMPLE 4

Using image analysis luster apparatus, we examined the optical properties of hair treated with two different compositions of vinyl pyrrolidone/vinyl carbazole copolymer. Luster analysis was conducted for (1) PVP K-30, (2) vinyl pyrrolidone/vinyl carbazole copolymer [90/10 wt %] of Example 1, and (3) vinyl pyrrolidone/vinyl carbazole copolymer [75/25 wt %] of Example 2. [R. McMullen and J. Jachowicz, Optical Properties of Hair: Effect of Treatments on Luster as Quantified by Image Analysis, J. Cosmet. Sci, 54, 335-351, (2003).]

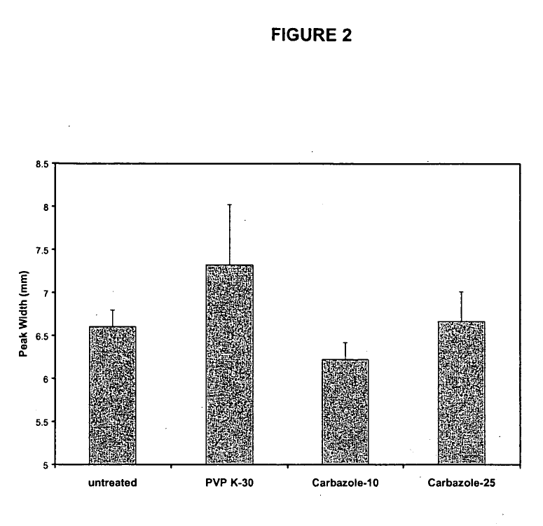

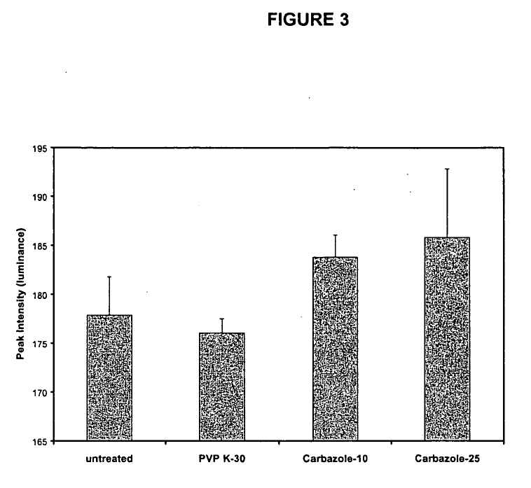

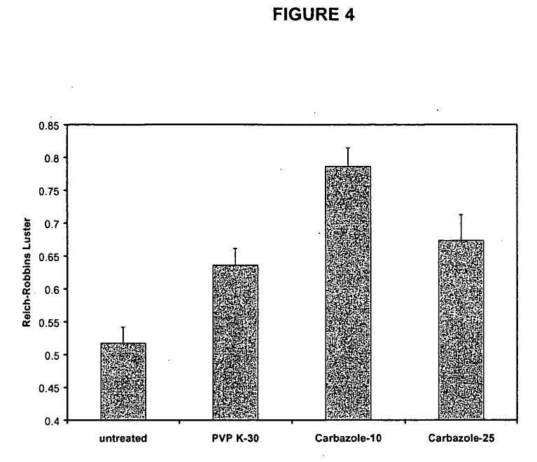

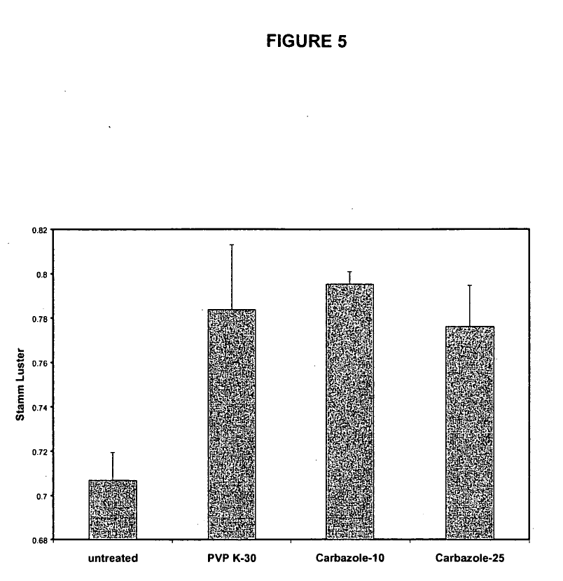

Various parameters have been used to determine luster and are summarized in FIGS. 2-5. The peak width (FIG. 2), serves as an indication of the sharpness of reflected light. Therefore, lower peak width values imply sharper reflection bands. We observed a narrower peak for hair treated with the copolymer containing 25% vinyl carbazole than we did for the rest of the materials. Peak intensity, summarized in FIG. 3, provides a measure of the amount of reflected light from hair in the reflection band region. As shown in the figure, there is an increase in peak intensity for hair treated with both copolymers of vinyl carbazole and vinyl pyrrolidone. We also utilize the Reich-Robbins (FIG. 4) and Stamm (FIG. 5) luster parameters to determine the efficacy of treatments. The Reich-Robbins luster increases when going from untreated hair to hair treated with PVP K-30. It is further increased when treated with the copolymer containing 10% vinyl carbazole. There is only a marginal difference between hair treated with PVP K-30 and vinyl pyrrolidone/vinyl carbazole copolymer [75/25 wt %] of Example 2 On the other hand, the Stamm luster reveals increases in hair treated with the polymers, however, differences among the polymers can not be discerned.

The luster parameters for vinyl pyrrolidone/vinyl carbazole copolymer compare favorably to previously determined parameters for VCL/VP/DMAEMA (Advantage LCA) and Phenyl Trimethicone. In relation to untreated hair the treated/untreated differences are as follows. Peak Intensity: 177±3/168±4 for Advantage LCA and 154±1/168±4 for Phenyl Trimethicone vs. 184±2/178±4 for co(VP-vinyl carbazole) [90/10 wt %]; Reich-Robbins Luster: 0.68±0.007/0.54±0.07 for Advantage LCA and 0.62±0.1/0.54±0.07 for Phenyl Trimethicone vs. 0.79±0.03/0.52±0.02 for co(VP-vinyl carbazole) [90/10 wt %]; Stamm Luster: 0.76±0.02/0.72±0.03 for Advantage LCA and 0.80±0.03/0.72±0.03 for Phenyl Trimethicone vs. 80±0.01/0.71±0.01 for co(VP-vinyl carbazole) [90/10 wt %].

In summary, the obtained results indicate that the reflective properties of vinyl pyrrolidone/vinyl carbazole copolymer [90/10 wt %] are superior to phenyl trimethicone, PVP, and equal or better than Advantage LCA.

While the copolymer of the invention is particularly useful for skin and hair care, it will be understood that it can be used in other personal care applications, such as an absorbent material in appropriate applications such as diapers, etc.

Thus, the copolymer of the invention is useful in combination with conventional cosmetic ingredients, such as conditioning agents, protecting agents, fixing agents, oxidizing agents, reducing agents, dyes and the like.

Any known conditioning agent is useful in the hair compositions of this invention. Conditioning agents function to improve the cosmetic properties of the hair, particularly softness, thickening, untangling, feel, and static electricity and may be in liquid, semi-solid, or solid form such as oils, waxes, or gums. Similarly, any known skin altering agent is useful in the compositions of this invention. Preferred conditioning agents include cationic polymers, cationic surfactants and cationic silicones.

Conditioning agents may be chosen from synthesis oils, mineral oils, vegetable oils, fluorinated or perfluorinated oils, natural or synthetic waxes, silicones, cationic polymers, proteins and hydrolyzed proteins, ceramide type compounds, cationic surfactants, fatty amines, fatty acids and their derivatives, as well as mixtures of these different compounds.

The synthesis oils include polyolefins, e.g., poly-α-olefins such as polybutenes, polyisobutenes and polydecenes. The polyolefins can be hydrogenated.

The mineral oils suitable for use in the compositions of the invention include hexadecane and oil of paraffin.

Suitable animal and vegetable oils include sunflower, corn, soy, avocado, jojoba, squash, raisin seed, sesame seed, walnut oils, fish oils, glycerol tricaprocaprylate, Purcellin oil or liquid jojoba.

Suitable natural or synthetic oils include eucalyptus, lavender, vetiver, litsea cubeba, lemon, sandalwood, rosemary, chamomile, savory, nutmeg, cinnamon, hyssop, caraway, orange, geranium, cade, and bergamot.

Suitable natural and synthetic waxes include carnauba wax, candelila wax, alfa wax, paraffin wax, ozokerite wax, vegetable waxes such as olive wax, rice wax, hydrogenated jojoba wax, absolute flower waxes such as black currant flower wax, animal waxes such as bees wax, modified bees wax (cerabellina), marine waxes and polyolefin waxes such as polyethylene wax. The cationic polymers that may be used as a conditioning agent in combination with the VP/VCZ copolymers according to the invention are those known to improve the cosmetic properties of hair treated by detergent compositions. The expression “cationic polymer” as used herein, indicates any polymer containing cationic groups and/or ionizable groups in cationic groups. The cationic polymers used generally have a number average molecular weight of which falls between about 500 and 5,000,000 and preferably between 1000 and 3,000,000.

The preferred cationic polymers are chosen from among those containing units including primary, secondary, tertiary, and/or quaternary amine groups that may either form part of the main polymer chain or a side chain.

Useful cationic polymers include known polyamine, polyaminoamide, and quaternary polyammonium types of polymers, such as:

(1) homopolymers and copolymers derived from acrylic or methacrylic esters or amides. The copolymers can contain one or more units derived from acrylamides, methacrylamides, diacetone acrylamides, acrylamides and acrylic or methacrylic acids or their esters, vinyllactams such as vinyl pyrrolidone or vinyl caprolactam, and vinyl esters. Specific examples include: copolymers of acrylamide and dimethylaminoethyl methacrylate quaternized with dimethyl sulfate or with an alkyl halide; copolymers of acrylamide and methacryloyloxyethyl trimethyl ammonium chloride; the copolymer of acrylamide and methacryloyloxyethyl trimethyl ammonium methosulfate; copolymers of vinyl pyrrolidone/dialkylaminoalkyl acrylate or methacrylate, optionally quaternized, such as the products sold under the name GAFQUAT by International Specialty Products; the dimethylaminoethyl methacrylate/vinyl caprolactam/vinyl pyrrolidone terpolymers, such as the product sold under the name GAFFIX® VC 713 by International Specialty Products; the vinyl pyrrolidone/dimethylaminopropyl methacrylamide copolymer, marketed under the name STYLEZE® CC 10 by International Specialty Products; and the vinyl pyrrolidone/quaternized dimethylaminopropyl methacrylamide copolymers such as the product sold under the name GAFQUAT® HS 100 by International Specialty Products.

(2) derivatives of cellulose ethers containing quaternary ammonium groups, such as hydroxy ethyl cellulose quaternary ammonium that has reacted with an epoxide substituted by a trimethyl ammonium group.

(3) derivatives of cationic cellulose such as cellulose copolymers or derivatives of cellulose grafted with a hydrosoluble quaternary ammonium monomer, as described in U.S. Pat. No. 4,131,576, such as the hydroxy alkyl cellulose, and the hydroxymethyl-, hydroxyethyl- or hydroxypropyl-cellulose grafted with a salt of methacryloylethyl trimethyl ammonium, methacrylamidopropyl trimethyl ammonium, or dimethyl diallyl ammonium.

(4) cationic polysaccharides such as described in U.S. Pat. Nos. 3,589,578 and 4,031,307, guar gums containing cationic trialkyl ammonium groups and guar gums modified by a salt, e.g., chloride of 2,3-epoxypropyl trimethyl ammonium.

(5) polymers composed of piperazinyl units and alkylene or hydroxy alkylene divalent radicals with straight or branched chains, possibly interrupted by atoms of oxygen, sulfur, nitrogen, or by aromatic or heterocyclic cycles, as well as the products of the oxidation and/or quaternization of such polymers.

(6) water-soluble polyamino amides prepared by polycondensation of an acid compound with a polyamine. These polyamino amides may be reticulated.

(7) derivatives of polyamino amides resulting from the condensation of polyalkoxylene polyamines with polycarboxylic acids followed by alkoxylation by bi-functional agents.

(8) polymers obtained by reaction of a polyalkylene polyamine containing two primary amine groups and at least one secondary amine group with a dioxycarboxylic acid chosen from among diglycolic acid and saturated dicarboxylic aliphatic acids having 3 to 8 atoms of carbon. Such polymers are described in U.S. Pat. Nos. 3,227,615 and 2,961,347.

(9) the cyclopolymers of alkyl diallyl amine or dialkyl diallyl ammonium such as the homopolymer of dimethyl diallyl ammonium chloride and copolymers of dimethyl diallyl ammonium chloride and acrylamide.

(10) quaternary diammonium polymers such as hexadimethrine chloride. Polymers of this type are described particularly in U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945, and 4,027,020.

(11) quaternary polyammonium polymers, including, for example, Mirapol® A 15, Mirapol® AD1, Mirapol® AZ1, and Mirapol® 175 products sold by Miranol.

(12) the quaternary polymers of vinyl pyrrolidone and vinyl imidazole such as the products sold under the names Luviquat® FC 905, FC 550, and FC 370 by BASF.

(13) quaternary polyamines.

(14) reticulated polymers known in the art.

Other cationic polymers that may be used within the context of the invention are cationic proteins or hydrolyzed cationic proteins, polyalkyleneimines such as polyethyleneimines, polymers containing vinyl pyridine or vinyl pyridinium units, condensates of polyamines and epichlorhydrins, quaternary polyurethanes, and derivatives of chitin.

Preferred cationic polymers are derivatives of quaternary cellulose ethers, the homopolymers and copolymers of dimethyl diallyl ammonium chloride, quaternary polymers of vinyl pyrrolidone and vinyl imidazole, and mixtures thereof.

The conditioning agent can be any silicone known by those skilled in the art to be useful as a conditioning agent. The silicones suitable for use according to the invention include polyorganosiloxanes that are insoluble in the composition. The silicones may be present in the form of oils, waxes, resins, or gums. They may be volatile or non-volatile. The silicones can be selected from polyalkyl siloxanes, polyaryl siloxanes, polyalkyl aryl siloxanes, silicone gums and resins, and polyorgano siloxanes modified by organofunctional groups, and mixtures thereof.

Suitable polyalkyl siloxanes include polydimethyl siloxanes with terminal trimethyl silyl groups or terminal dimethyl silanol groups (dimethiconol) and polyalkyl (C₁-C₂₀) siloxanes.

Suitable polyalkyl aryl siloxanes include polydimethyl methyl phenyl siloxanes and polydimethyl diphenyl siloxanes, linear or branched.

The silicone gums suitable for use herein include polydiorganosiloxanes preferably having a number-average molecular weight between 200,000 and 1,000,000, used alone or mixed with a solvent. Examples include polymethyl siloxane, polydimethyl siloxane/methyl vinyl siloxane gums, polydimethyl siloxane/diphenyl siloxane, polydimethyl siloxane/phenyl methyl siloxane and polydimethyl siloxane/diphenyl siloxane/methyl vinyl siloxane.

Suitable silicone resins include silicones with a dimethyl/trimethyl siloxane structure and resins of the trimethyl siloxysilicate type.

The organo-modified silicones suitable for use in the invention include silicones such as those previously defined and containing one or more organofunctional groups attached by means of a hydrocarbon radical and grafted siliconated polymers. Particularly preferred are amino functional silicones.

The silicones may be used in the form of emulsions, nano-emulsions, or micro-emulsions.

The conditioning agent can be a protein or hydrolyzed cationic or non-cationic protein. Examples of these compounds include hydrolyzed collagens having triethyl ammonium groups, hydrolyzed collagens having trimethyl ammonium and trimethyl stearyl ammonium chloride groups, hydrolyzed animal proteins having trimethyl benzyl ammonium groups (benzyltrimonium hydrolyzed animal protein), hydrolyzed proteins having groups of quaternary ammonium on the polypeptide chain, including at least one C₁-C₁₈ alkyl.

Hydrolyzed proteins include Croquat L, in which the quaternary ammonium groups include a C₁₂ alkyl group, Croquat M, in which the quaternary ammonium groups include C₁₀-C₁₈ alkyl groups, Croquat S in which the quaternary ammonium groups include a C₁₈ alkyl group and Crotein Q in which the quaternary ammonium groups include at least one C₁-C₁₈ alkyl group. These products are sold by Croda.

The conditioning agent can comprise quaternized vegetable proteins such as wheat, corn, or soy proteins such as cocodimonium hydrolyzed wheat protein, laurdimonium hydrolyzed wheat protein and steardimonium hydrolyzed wheat protein.

According to the invention, the conditioning agent can be a ceramide type of compound such as a ceramide, a glycoceramide, a pseudoceramide, or a neoceramide. These compounds can be natural or synthetic. Compounds of the ceramide type are, for example, described in Patents pending DE4424530, DE4424533, DE4402929, DE4420736, WO95/23807, WO94/07844, EP-A-0646572, WO95/16665, FR-2 673 179, EP-A-0227994, WO 94/07844, WO 94/24097, and WO 94/10131. Ceramide type compounds useful herein include 2-N-linoleoyl amino-octadecane-1,3-diol, 2-N-oleoyl amino-octadecane-1,3-diol, 2-N-palmitoyl amino-octadecane-1,3-diol, 2-N-stearoyl amino-octadecane-1,3-diol, 2-N-behenoyl amino-octadecane-1,3-diol, 2-N-[2-hydroxy-palmitoyl]-amino-octadecane-1,3-diol, 2-N-stearoyl amino-octadecane-1,3,4-triol, N-stearoyl phytosphingosine, 2-N-palmitoyl amino-hexadecane-1,3-diol, bis-(N-hydroxy ethyl N-cetyl) malonamide, N(2-hydroxy ethyl)-N-(3-cetoxyl-2-hydroxy propyl) amide of cetylic acid, N-docosanoyl N-methyl-D-glucamine and mixtures of such compounds.

The conditioning agent can be a cationic surfactant such as a salt of a primary, secondary, or tertiary fatty amine, optionally polyoxyalkylenated, a quaternary ammonium salt, a derivative of imadazoline, or an amine oxide. Suitable examples include mono-, di-, or tri-alkyl quaternary ammonium compounds with a counterion such as a chloride, methosulfate, tosylate, etc. including, but not limited to, cetrimonium chloride, dicetyldimonium chloride, behentrimonium methosulfate, and the like. The presence of a quaternary ammonium compound in conjunction with the polymer described above reduces static and enhances combing of hair in the dry state. The polymer also enhances the deposition of the quaternary ammonium compound onto the hair substrate thus enhancing the conditioning effect of hair.

The conditioning agent can be any fatty amine known to be useful as a conditioning agent; e.g. dodecyl, cetyl or stearyl amines, such as stearamidopropyl dimethylamine.

The conditioning agent can be a fatty acid or derivatives thereof known to be useful as conditioning agents. Suitable fatty acids include myristic acid, palmitic acid, stearic acid, behenic acid, oleic acid, linoleic acid, and isostearic acid. The derivatives of fatty acids include carboxylic ester acids including mono-, di-, tri- and tetra-carboxylic acids.

The conditioning agent can be a fluorinated or perfluorinated oil. Fluorinated oils include perfluoropolyethers described in EP-A-486135 and the fluorohydrocarbon compounds described in WO 93/11103. The fluoridated oils may also be fluorocarbons such as fluoramines, e.g., perfluorotributylamine, fluoridated hydrocarbons, such as perfluorodecahydronaphthalene, fluoroesters, and fluoroethers.

Of course, mixtures of two or more conditioning agents can be used.

The conditioning agent or agents can be present in an amount of 0.001% to 20%, preferably from 0.01% to 10%, and even more preferably from 0.1% to 3% by weight based on the total weight of the final composition.

The composition of the invention can contain one or more protecting agents to prevent or limit the degrading effects of natural physical and/or chemical assaults on the keratinous materials.

The protecting agent can be chosen from hydrosoluble, liposoluble and water-insoluble UV filters, antiradical agents, antioxidants, vitamins and pro-vitamins. The above-described cationic polymer enhances the deposition of these materials onto the hair or skin substrate enhancing protection of hair to UV damage.

Organic UV filters (systems that filter out UV rays) can be chosen from among hydrosoluble or liposoluble filters, whether siliconated or nonsiliconated, and mineral oxide particles, the surface of which may be treated.

Hydrosoluble organic UV filters may be chosen from para-amino benzoic acid and its salts, anthranilic acid and its salts, salicylic acid and its salts, hydroxy cinnamic acid and its salts, sulfonic derivatives of benzothiazoles, benzimidizoles, benzoxazoles and their salts, sulfonic derivatives of benzophenone and their salts, sulfonic derivatives of benzylidene camphor and their salts, derivatives of benzylidene camphor substituted by a quaternary amine and their salts, derivatives of phthalydene-camphosulfonic acids and their salts, sulfonic derivatives of benzotriazole, and mixtures thereof.

Hydrophilic polymers which have light-protective qualities against UV rays can be used. These include polymers containing benzylidene camphor and/or benzotriazole groups.

Suitable liposoluble organic UV filters include derivatives of para-aminobenzoic acid, such as the esters or amides of para-aminobenzoic acid; derivatives of salicylic acid; derivatives of benzophenone; derivatives of dibenzoyl methane; derivatives of diphenyl acrylates; derivatives of benzofurans; UV filter polymers containing one or more silico-organic residues; esters of cinnamic acid; derivatives of camphor; derivatives of trianilino-s-triazine; the ethylic ester urocanic acid; benzotriazoles; derivatives of hydroxy phenyl triazine; bis-resorcinol-dialkyl amino triazine; and mixtures thereof.

The liposoluble (or lipophilic) organic UV filter according to the invention can be chosen from octyl salicylate; 4-tert-butyl-4′-methoxy dibenzoyl methane; octocrylene; 4-methoxy cinnamate; 2-ethylhexyl [2-ethylhexyl 4-methoxycinnamate]; and 2-(2H-benzotriazol-2-yl)-4-methyl-6-[2-methyl-3-[1,3,3,3-tetramethyl-1-[(trimethyl silyl)oxy] disiloxanyl]propynyl] phenol.

Other UV filters particularly preferred for use herein are derivatives of benzophenones such as 2-hydroxy-4-methoxy benzophenone-5-sulfonic acid, 2-hydroxy-4-methoxy benzophenone, derivatives of benzalmalonates such as poly dimethyl/methyl (3(4-(2,2-bis-ethoxy carbonyl vinyl)-phenoxy)-propenyl) siloxane, derivatives of benzylidene camphor such as b-b′camphosulfonic [1-4 divinylbenzene] acid and derivatives of benzimidazole such as 2-phenyl-benzimidazol-5-sulfonic acid.

Water-insoluble UV filters include various mineral oxides. The mineral oxides may be selected from among titanium oxides, zinc oxides, and cerium oxides. The mineral oxides can be used in the form of ultrafine nanoparticles.

Preferred UV filters include Escalol HP-610 (dimethylpabamido propyl laurdimonium tosylate and propylene glycol stearate) and Crodasorb HP (polyquaternium 59).

The antioxidants or antiradical agents can be selected from phenols such as BHA (tert-butyl-4-hydroxy anisole), BHT (2,6-di-tert-butyl-p-cresol), TBHQ (tert-butyl hydroquinone), polyphenols such as proanthocyanodic oligomers, flavonoids, hindered amines such as tetra amino piperidine, erythorbic acid, polyamines such as spermine, cysteine, glutathione, superoxide dismutase, and lactoferrin.

The vitamins can be selected from ascorbic acid (vitamin C), vitamin E, vitamin E acetate, vitamin E phosphate, B vitamins such as B3 and B5, vitamin PP, vitamin A, and derivatives thereof. The provitamins can be selected from panthenol and retinol.

The protecting agent can be present in an amount 0.001% to 20% by weight, preferably from 0.01% to 10% by weight, and more preferably 0.1 to 5% by weight of the total weight of the final composition.

The composition of the invention can contain a fixing agent.

The fixing agent can be an anionic polymer chosen from polymers containing carboxylic units derived from unsaturated carboxylic mono- or polyacids of the formula: 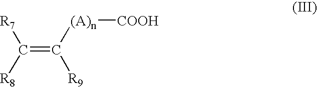
in which n is a whole number from 0 to 10, A₁ denotes a methylene group, optionally bonded to the carbon atom of the unsaturated group or to a neighboring methylene group when n is greater than 1 by means of a heteroatom like oxygen or sulfur, R₇ denotes a hydrogen atom, a phenyl or benzyl group, R₈ denotes a hydrogen atom, a lower alkyl or carboxyl group, R₉ denotes a hydrogen atom, a lower alkyl group, a —CH₂—COOH, phenyl or benzyl group and polymers containing units derived from sulfonic acid like vinylsulfonic, styrenesulfonic, acrylamidoalkylsulfonic units.

The fixing agent can be an amphoteric polymer chosen from the polymer containing recurring units derived from:
a) at least one monomer chosen from acrylamides or methacrylamides substituted on the nitrogen with an alkyl radical,
b) at least one acid copolymer containing one or more reactive carboxyl groups, and
c) at least one basic comonomer, such as esters with primary, secondary, tertiary, and quaternary amino substituents of acrylic and methacrylic acids and the product of quaternization of dimethylaminoethyl methacrylate with dimethyl or diethyl sulfate.

The fixing agent can be a nonionic polymer chosen from polyalkyloxazolines; vinyl acetate homopolymers; vinyl acetate and acrylic ester copolymers; vinyl acetate and ethylene copolymers; vinyl acetate and maleic ester copolymers; polyethylene and maleic anhydride copolymers; homopolymers of alkyl acrylates; homopolymers of alkyl methacrylates; copolymers of acrylic esters; copolymers of alkyl acrylates and alkyl methacrylates; copolymers of acrylonitrile and a nonionic monomer chosen from among butadiene and alkyl (meth)acrylates; copolymers of alkyl acrylate and urethane; and polyamides.

The fixing polymer can be a functionalized or unfunctionalized, silicone or non-silicone polyurethane.

The fixing polymer can be a polymer of the grafted silicone type containing a polysiloxane portion and a portion consisting of a nonsilicone organic chain, with one of the two portions forming the main chain of the polymer, and with the other being grafted onto said main chain.

The fixing agent can be present in the composition in a relative weight concentration between 0.1 and 10%, preferably 0.5 and 5%.

The composition of the invention can contain an oxidizing agent. The oxidizing agent can be chosen from the group of hydrogen peroxide, urea peroxide, alkali metal bromates, ferricyanides, persalts, and redox enzymes, optionally with their respective donor or cofactor. In a particularly preferred embodiment, the oxidizing agent is hydrogen peroxide. The oxidizing agent can be a solution of oxygenated water whose titer varies from 1 to 40 volumes.

The composition of the invention can contain at least one reducing agent in amounts from 0.01 to 30 wt %, preferably 0.05 to 20 wt % of the total weight of the composition. The reducing agents useful in the practice of this invention can be selected from thiols, like cysteine, thioglycolic acid, thiolactic acid, their salts and esters, cysteamine, and its salts or sulfites. In the case of compositions intended for bleaching, ascorbic acid, its salts and its esters, erythorbic acid, its salts and its esters, and sulfinates, like sodium hydroxymethanesulfinate can be used.

The composition of the invention can contain a dye selected from the group consisting of neutral acid or cationic nitrobenzene dyes, neutral acid or cationic azo dyes, quinone dyes, neutral, acid or cationic anthraquinone dyes, azine dyes, triarylmethane dyes, indoamine dyes and natural dyes. The dye or dyes can be present in a concentration from 0.001 to 20% and preferably 0.005 to 10 wt % based on the total weight of the composition.

The composition of the invention can contain at least one amphoteric polymer or a cationic polymer different from the cationic poly(vinyllactam) defined above. Suitable cationic polymers include a poly(quaternary ammonium) consisting of recurrent units corresponding to the following formulae (W) and (U):

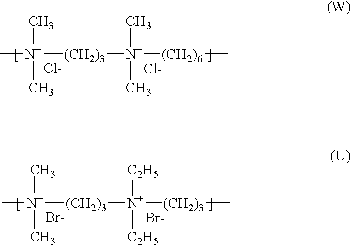

Suitable amphoteric polymers include a copolymer containing at least one acrylic acid and a dimethyldiallyammonium salt as a monomer. The cationic or amphoteric polymer or polymers can be present in an amount of 0.01 to 10%, preferably 0.05 to 5%, and more preferably 0.1 to 3% by weight of the total weight of the composition.

In addition, the compositions according to the invention advantageously include at least one surfactant, which can be present in an amount of 0.1% and 60% preferably 1% and 40%, and more preferably 5% and 30% by weight based on the total weight of the composition. The surfactant may be chosen from among anionic, amphoteric, or non-ionic surfactants, or mixtures of them known to be useful in personal care compositions.

The composition of the invention can contain one or more additional cosmetically acceptable additives chosen from conditioning agents, protecting agents, such as, for example, hydrosoluble, liposoluble and water-insoluble UV filters, antiradical agents, antioxidants, vitamins and pro-vitamins, fixing agents, oxidizing agents, reducing agents, dyes, cleansing agents, anionic, cationic, nonionic and amphoteric surfactants, thickeners, perfumes, pearlizing agents, stabilizers, pH adjusters, filters, preservatives, hydroxy acids, cationic and nonionic polyether associative polyurethanes, polymers other than the cationic polymer described herein, vegetable oils, mineral oils, synthetic oils, polyols such as glycols and glycerol, silicones, aliphatic alcohols, colorants, bleaching agents, highlighting agents and sequestrants. These additives are present in the composition according to the invention in proportions that may range from 0 to 20% by weight in relation to the total weight of the composition. The precise amount of each additive may be easily determined by an expert in the field according to its nature and its function.

Additional thickeners or viscosity increasing agents may be included in the composition of the invention, such as:

Acetamide MEA Acrylamide/Ethalkonium Chloride Acrylate Copolymer

Acrylamide/Ethyltrimonium Chloride Acrylate/Ethalkonium Chloride Acrylate

Copolymer

Acrylamides Copolymer

Acrylamide/Sodium Acrylate Copolymer

Acrylamide/Sod ium Acryloyldimethyltaurate Copolymer

Acrylates/Acetoacetoxyethyl Methacrylate Copolymer

Acrylates/Beheneth-25 Methacrylate Copolymer

Acrylates/C10-30 Alkyl Acrylate Crosspolymer

Acrylates/Ceteth-20 Itaconate Copolymer

Acrylates/Ceteth-20 Methacrylate Copolymer

Acrylates/La ureth-25 Methacrylate Copolymer

Acrylates/Palmeth-25 Acrylate Copolymer

Acrylates/Palmeth-25 Itaconate Copolymer

Acrylates/Steareth-50 Acrylate Copolymer

Acrylates/Steareth-20 Itaconate Copolymer

Acrylates/Steareth-20 Methacrylate Copolymer

Acrylates/Stearyl Methacrylate Copolymer

AcrylatesNinyl Isodecanoate Crosspolymer

Acrylic Acid/Acrylonitrogens Copolymer

Adipic Acid/Methyl DEA Crosspolymer

Agar

Agarose

Alcaligenes Polysaccharides

Algin

Alginic Acid

Almondamide DEA

Almondamidopropyl Betaine

Aluminum/Magnesium Hydroxide Stearate

Ammonium Acrylates/Acrylonitrogens Copolymer

Ammonium Acrylates Copolymer

Ammonium AcryloyidimethyltaurateNinyl Formamide Copolymer

Ammonium AcryloyidimethyltaurateNP Copolymer

Ammonium Alginate

Ammonium Chloride

Ammonium Polyacryloyldimethyl Taurate

Ammonium Sulfate

Amylopectin

Apricotamide DEA

Apricotamidopropyl Betaine

Arachidyl Alcohol

Arachidyl Glycol

Arachis Hypogaea (Peanut) Flour

Ascorbyl Methylsilanol Pectinate

Astragalus Gummifer Gum

Attapulgite

Avena Sativa (Oat) Kernel Flour

Avocadamide DEA

Avocadamidopropyl Betaine

Azelamide MEA

Babassuamide DEA

Babassuamide MEA

Babassuamidopropyl Betaine

Behenamide DEA

Behenamide MEA

Behenamidopropyl Betaine

Behenyl Betaine

Bentonite

Butoxy Chitosan

Caesalpinia Spinosa Gum

Calcium Alginate

Calcium Carboxymethyl Cellulose

Calcium Carrageenan

Calcium Chloride

Calcium Potassium Carbomer

Calcium Starch Octenylsuccinate

C20-40 Alkyl Stearate

Canolamidopropyl Betaine

Capramide DEA

Capryl/Capramidopropyl Betaine

Carbomer

Carboxybutyl Chitosan

Carboxymethyl Cellulose Acetate Butyrate

Carboxymethyl Chitin

Carboxymethyl Chitosan

Carboxymethyl Dextran

Carboxymethyl Hydroxyethylcellulose

Carboxymethyl Hydroxypropyl Guar

Carnitine

Cellulose Acetate Propionate Carboxylate

Cellulose Gum

Ceratonia Siliqua Gum

Cetearyl Alcohol

Cetyl Alcohol

Cetyl Babassuate

Cetyl Betaine

Cetyl Glycol

Cetyl Hydroxyethylcellulose

Chimyl Alcohol

Cholesterol/HDI/Pullulan Copolymer

Cholesteryl Hexyl Dicarbamate Pullulan

Citrus Aurantium Dulcis (Orange) Peel Extract

Cocamide DEA

Cocamide MEA

Cocamide MIPA

Cocamidoethyl Betaine

Cocamidopropyl Betaine

Cocamidopropyl Hydroxysultaine

Coco-Betaine

Coco-Hydroxysultaine

Coconut Alcohol

Coco/Oleamidopropyl Betaine

Coco-Sultaine

Cocoyl Sarcosinamide DEA

Cornamide/Cocamide DEA

Cornamide DEA

Croscarmellose

Crosslinked Bacillus/Glucose/Sodium Glutamate Ferment

Cyamopsis Tetragonoloba (Guar) Gum

Decyl Alcohol

Decyl Betaine

Dehydroxanthan Gum

Dextrin

Dibenzylidene Sorbitol

Diethanolaminooleamide DEA

Diglycol/CHDM/lsophthalates/SIP Copolymer

Dihydroabietyl Behenate

Dihydrogenated Tallow Benzylmonium Hectorite

Dihydroxyaluminum Aminoacetate

Dimethicone/PEG-10 Crosspolymer

Dimethicone/PEG-15 Crosspolymer

Dimethicone Propyl PG-Betaine

Dimethylacrylamide/Acrylic Acid/Polystyrene Ethyl Methacrylate Copolymer

Dimethylacrylamide/Sodium Acryloyldimethyltaurate Crosspolymer

Disteareth-100 IPDI

DMAPA Acrylates/Acrylic Acid/Acrylonitrogens Copolymer

Erucamidopropyl Hydroxysultaine

Ethylene/Sodium Acrylate Copolymer

Gelatin

Gellan Gum

Glyceryl Alginate

Glycine Soja (Soybean) Flour

Guar Hydroxypropyltrimonium Chloride

Hectorite

Hyaluronic Acid

Hydrated Silica

Hydrogenated Potato Starch

Hydrogenated Tallow

Hydrogenated Tallowamide DEA

Hydrogenated Tallow Betaine

Hydroxybutyl Methylcellulose

Hydroxyethyl Acrylate/Sodium Acryloyldimethyl Taurate Copolymer

Hydroxyethylcellulose

Hydroxyethyl Chitosan

Hydroxyethyl Ethylcellulose

Hydroxyethyl Stearamide-MIPA

Hydroxylauryl/Hydroxymyristyl Betaine

Hydroxypropylcellulose

Hydroxypropyl Chitosan

Hydroxypropyl Ethylenediamine Carbomer

Hydroxypropyl Guar

Hydroxypropyl Methylcellulose

Hydroxypropyl Methylcellulose Stearoxy Ether

Hydroxypropyl Starch

Hydroxypropyl Starch Phosphate

Hydroxypropyl Xanthan Gum

Hydroxystearamide MEA

Isobutylene/Sodium Maleate Copolymer

Isostearamide DEA

Isostearamide MEA

Isostearamide MIPA

Isostearamidopropyl Betaine

Lactamide MEA

Lanolinamide DEA

Lauramide DEA

Lauramide MEA

Lauramide MIPA

Lauramide/Myristamide DEA

Lauramidopropyl Betaine

Lauramidopropyl Hyd roxysultaine

Laurimino Bispropanediol

Lauryl Alcohol

Lauryl Betaine

Lauryl Hydroxysultaine

Lauryl/Myristyl Glycol Hydroxypropyl Ether

Lauryl Sultaine

Lecithinamide DEA

Linoleamide DEA

Linoleamide MEA

Linoleamide MIPA

Lithium Magnesium Silicate

Lithium Magnesium Sodium Silicate

Macrocystis Pyrifera (Kelp)

Magnesium Alginate

Magnesium/Aluminum/Hydroxide/Carbonate

Magnesium Aluminum Silicate

Magnesium Silicate

Magnesium Trisilicate

Methoxy PEG-22/Dodecyl Glycol Copolymer

Methylcellulose

Methyl Ethylcellulose

Methyl Hydroxyethylcellulose

Microcrystalline Cellulose

Milkamidopropyl Betaine

Minkamide DEA

Minkamidopropyl Betaine

MIPA-Myristate

Montmorillonite

Moroccan Lava Clay

Myristamide DEA

Myristamide MEA

Myristamide MIPA

Myristamidopropyl Betaine

Myristamidopropyl Hydroxysultaine

Myristyl Alcohol

Myristyl Betaine

Nafto Gum

Nonoxynyl Hyd roxyethylcellulose

Oatamide MEA

Oatamidopropyl Betaine

Octacosanyl Glycol Isostearate

Octadecene/MA Copolymer

Oleamide DEA

Oleamide MEA

Oleamide MIPA

Oleamidopropyl Betaine

Oleamidopropyl Hydroxysultaine

Oleyl Betaine

Olivamide DEA

Olivamidopropyl Betaine

Oliveamide MEA

Palmamide DEA

Palmamide MEA

Palmamide MIPA

Palmamidopropyl Betaine

Palmitamide DEA

Palmitamide MEA

Palmitamidopropyl Betaine

Palm Kernel Alcohol

Palm Kernelamide DEA

Palm Kernelamide MEA

Palm Kernelamide MIPA

Palm Kernelamidopropyl Betaine

Peanutamide MEA

Peanutamide MIPA

Pectin

PEG-800

PEG-Crosspolymer

PEG-150/Decyl Alcohol/SMDI Copolymer

PEG-175 Diisostearate

PEG-190 Distearate

PEG-15 Glyceryl Tristearate

PEG-140 Glyceryl Tristearate

PEG-240/HDI Copolymer Bis-Decyltetradeceth-20 Ether

PEG-100/IPDI Copolymer

PEG-180/Laureth-50/TMMG Copolymer

PEG-10/Lauryl Dimethicone Crosspolymer

PEG-15/Lauryl Dimethicone Crosspolymer

PEG-2M

PEG-5M

PEG-7M

PEG-9M

PEG-14M

PEG-20M

PEG-23M

PEG-25M

PEG-45M

PEG-65M

PEG-90M

PEG-115M

PEG-160M

PEG-180M

PEG-120 Methyl Glucose Trioleate

PEG-180/Octoxynol-40/TMMG Copolymer

PEG-150 Pentaerythrityl Tetrastearate

PEG-4 Rapeseedamide

PEG-150/Stearyl Alcohol/SMDI Copolymer

Phaseolus Angularis Seed Powder

Polianthes Tuberosa Extract

Polyacrylate-3

Polyacrylic Acid

Polycyclopentadiene

Polyether-1

Polyethylene/Isopropyl Maleate/MA Copolyol

Polyglyceryl-3 Disiloxane Dimethicone

Polyglyceryl-3 Polydimethylsiloxyethyl Dimethicone

Polymethacrylic Acid

Polyquaternium-52

Polyvinyl Alcohol

Potassium Alginate

Potassium Aluminum Polyacrylate

Potassium Carbomer

Potassium Carrageenan

Potassium Chloride

Potassium Palmate

Potassium Polyacrylate

Potassium Sulfate

Potato Starch Modified

PPG-2 Cocamide

PPG-1 Hydroxyethyl Caprylamide

PPG-2 Hydroxyethyl Cocamide

PPG-2 Hydroxyethyl Coco/isostearamide

PPG-3 Hydroxyethyl Soyamide

PPG-14 Laureth-60 Hexyl Dicarbamate

PPG-14 Laureth-60 Isophoryl Dicarbamate

PPG-14 Palmeth-60 Hexyl Dicarbamate

Propylene Glycol Alginate

PVP/Decene Copolymer

PVP Montmorillonite

Pyrus Cydonia Seed

Pyrus Malus (Apple) Fiber

Rhizobian Gum

Ricebranamide DEA

Ricinoleamide DEA

Ricinoleamide MEA

Ricinoleamide MIPA

Ricinoleamidopropyl Betaine

Ricinoleic Acid/Adipic Acid/AEEA Copolymer

Rosa Multiflora Flower Wax

Sclerotium Gum

Sesamide DEA

Sesamidopropyl Betaine

Sodium Acrylate/Acryloyidimethyl Taurate Copolymer

Sodium Acrylates/Acrolein Copolymer

Sodium Acrylates/Acrylonitrogens Copolymer

Sodium Acrylates Copolymer

Sodium Acrylates Crosspolymer

Sodium Acrylate/Sodium Acrylamidomethylpropane Sulfonate Copolymer

Sodium AcrylatesNinyl Isodecanoate Crosspolymer

Sodium Acrylate/Vinyl Alcohol Copolymer

Sodium Carbomer

Sodium Carboxymethyl Chitin

Sodium Carboxymethyl Dextran

Sodium Carboxymethyl Beta-Glucan

Sodium Carboxymethyl Starch

Sodium Carrageenan

Sodium Cellulose Sulfate

Sodium Chloride

Sodium Cyclodextrin Sulfate

Sodium Hydroxypropyl Starch Phosphate

Sodium Isooctylene/MA Copolymer

Sodium Magnesium Fluorosilicate

Sodium Oleate

Sodium Palmitate

Sodium Palm Kernelate

Sodium Polyacrylate

Sodium Polyacrylate Starch

Sodium Polyacryloyldimethyl Taurate

Sodium Polygamma-Glutamate

Sodium Polymethacrylate

Sodium Polystyrene Sulfonate

Sodium Silicoaluminate

Sodium Starch Octenylsuccinate

Sodium Stearate

Sodium Stearoxy PG-Hydroxyethylcellulose Sulfonate

Sodium Styrene/Acrylates Copolymer

Sodium Sulfate

Sodium Tallowate

Sodium Tauride Acrylates/Acrylic Acid/Acrylonitrogens Copolymer

Sodium Tocopheryl Phosphate

Solanum Tuberosum (Potato) Starch

Soyamide DEA

Soyamidopropyl Betaine

Starch/Acrylates/Acrylamide Copolymer

Starch Hydroxypropyltrimonium Chloride

Stearamide AMP

Stearamide DEA

Stearamide DEA-Distearate

Stearamide DIBA-Stearate

Stearamide MEA

Stearamide MEA-Stearate

Stearamide MIPA

Stearamidopropyl Betaine

Steareth-60 Cetyl Ether

Steareth-100/PEG-136/HDI Copolymer

Stearyl Alcohol

Stearyl Betaine

Sterculia Urens Gum

Synthetic Fluorphlogopite

Tallamide DEA

Tallow Alcohol

Tallowamide DEA

Tallowamide MEA

Tallowamidopropyl Betaine

Tallowamidopropyl Hydroxysultaine

Tallowamine Oxide

Tallow Betaine

Tallow Dihydroxyethyl Betaine

Tamarindus Indica Seed Gum

Tapioca Starch

TEA-Alginate

TEA-Carbomer

TEA-Hydrochloride

Trideceth-2 Carboxamide MEA

Tridecyl Alcohol

Triethylene Glycol Dibenzoate

Trimethyl Pentanol Hydroxyethyl Ether

Triticum Vulgare (Wheat) Germ Powder

Triticum Vulgare (Wheat) Kernel Flour

Triticum Vulgare (Wheat) Starch

Tromethamine Acrylates/Acrylonitrogens Copolymer

Tromethamine Magnesium Aluminum Silicate

Undecyl Alcohol

Undecylenamide DEA

Undecylenamide MEA

Undecylenamidopropyl Betaine

Welan Gum

Wheat Germamide DEA

Wheat Germamidopropyl Betaine

Xanthan Gum

Yeast Beta-Glucan

Yeast Polysaccharides and Zea Mays (Corn) Starch.

Preferred thickeners or viscosity increasing agents include Carbomer, Aculyn and Stabileze, e.g. crosslinked acrylic acid, crosslinked poly(methylvinyl ether/maleic anhydride) copolymer, acrylamides, carboxymethyl cellulose and the like.

The compositions according to the invention may be used to wash and treat keratinous material such as hair, skin, eyelashes, eyebrows, fingernails, lips, and hairy skin.

The compositions according to the invention can be detergent compositions such as shampoos, bath gels, and bubble baths. In this mode, the compositions will comprise a generally aqueous washing base. The surfactant or surfactants that form the washing base may be chosen alone or in blends, from known anionic, amphoteric, or non-ionic surfactants. The quantity and quality of the washing base must be sufficient to impart a satisfactory foaming and/or detergent value to the final composition. The washing base can be from 4% to 50% by weight, preferably from 6% to 35% by weight, and even more preferentially from 8% to 25% by weight of the total weight of the final composition.

The pH of the composition applied to the keratinous material is generally between 2 and 12. It is preferably between 3 and 8, and may be adjusted to the desired value by means of acidifying or alkalinizing agents that are well-known in the state of the art in compositions applied to keratinous materials. Thus, the composition of the invention can contain at least one alkalizing or acidifying agent in amounts from 0.01 to 30 wt % of the total weight of the composition.

The alkalizing agent can be chosen from ammonia, alkali carbonates, alkanolamines, like mono-, di- and triethanolamines, as well as their derivatives, hydroxyalkylamines and ethoxylated and/or propoxylated ethylenediamines, sodium or potassium hydroxides and compounds of the following formula (XIX): 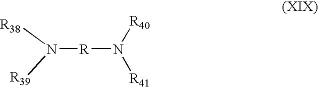
in which R is a propylene residue optionally substituted with an hydroxyl group or a C₁-C₄ alkyl radical; R₃₈, R₃₉, R₄₀ and R₄₁, identical or different, represent a hydrogen atom, a C₁-C₄ alkyl radical or C₁-C₄ hydroxyalkyl radical.

The acidifying agent can be chosen from mineral or organic acids, like hydrochloric acid, orthophosphoric acid, carboxylic acids like tartaric acid, citric acid, or lactic acid, or sulfonic acids and the like.

The physiological and cosmetically acceptable medium may consist exclusively of water, a cosmetically acceptable solvent, or a blend of water and a cosmetically acceptable solvent, such as a lower alcohol composed of C₁ to C₄, such as ethanol, isopropanol, t-butanol, n-butanol, alkylene glycols such as propylene glycol, and glycol ethers. However, the compositions of the invention can be anhydrous.

Generally the present cosmetic compositions are prepared by simple mixing procedures well known in the art.

The invention also has as its object a process for treating keratinous material including the skin or hair, characterized in that it consists of applying to skin or keratinous materials a cosmetic composition as described above, and then eventually rinsing it with water. Accordingly, the process according to the invention makes it possible to maintain the hairstyle, treatment, care, washing, or make-up removal of the skin, the hair, and any other keratinous material.

The compositions according to the invention may also take the form of after-shampoo compositions, to be rinsed off or not, for permanents, straightening, waving, dyeing, or bleaching, or the form of rinse compositions to be applied before or after dyeing, bleaching, permanents, straightening, relaxing, waving or even between the two stages of a permanent or straightening process.

The compositions of the invention may also take the form of skin-washing compositions, and particularly in the form of solutions or gels for the bath or shower, or of make-up removal products.

The compositions of the invention may also be in the form of aqueous or hydro-alcoholic solutions for skin and/or hair care.

The compositions described herein are useful in products for personal care, including, but mot limited to, gels, lotions, glazes, glues, mousses, sprays, fixatives, shampoos, conditioners, 2n1 shampoos, temporary hair dyes, semi-permanent hair dyes, permanent hair dyes, straighteners, permanent waves, relaxers, creams, putties, waxes, pomades, moisturizers, mascaras, lip balms and foam enhancers.

What is claimed is:

1. A copolymer of a vinyl lactam and vinyl carbazole.

2. A copolymer according to claim 1 wherein said vinyl lactam is vinyl pyrrolidone.

3. A copolymer according to claim 1 wherein said vinyl lactam is vinyl caprolactam.

4. A copolymer according to claim 1 which is water and/or alcohol soluble.

5. A water soluble copolymer of claim 1 which includes about 10% w/w of vinyl carbazole.

6. An alcohol soluble copolymer of claim 1 which includes about 25% w/w of vinyl carbazole.

7. A copolymer according to claim 1 which exhibits fluorescence.

8. A copolymer according to claim 1 which includes one or more monomers selected from dimethylaminopropyl(meth)acrylamide (DMAPMA), C₁-C₁₈-quaternized DMAPMA, dimethylaminoethyl(meth)acrylate (DMAEMA), C₁-C₁₈-quaternized DMAEMA and acrylamide.

9. A personal care composition including the copolymer of claim 1.

10. A personal care composition according to claim 9 which is a skin or hair care composition.

11. A personal care composition according to claim 10 which exhibits fluorescence and/or UV absorption.
